(19) United States
(12) Patent Application Publication
(10) Pub. No.: US 2013/0023973 A1
(43) Pub. Date: Jan. 24, 2013

(54) HIGH DENSITY IMPLANTABLE CONNECTOR

(71) Applicant: Neurostream Technologies General Partnership, Quebec (CA)

(72) Inventors: Martin Richard, Montreal (CA); Pierre-Etienne Boiteau, Quebec (CA)

(73) Assignee: NEUROSTREAM TECHNOLOGIES GENERAL PARTNERSHIP, Quebec (CA)

(21) Appl. No.: 13/631,267

(22) Filed: Sep. 28, 2012

Related U.S. Application Data
(63) Continuation of application No. 13/413,409, filed on Mar. 6, 2012.
Application No. 13/413,409 is a continuation of application No. 13/213,060, filed on Aug. 18, 2011, now abandoned.
Application No. 13/213,060 is a continuation of application No. 12/979,997, filed on Dec. 28, 2010, now abandoned.
Application No. 12/979,997 is a continuation of application No. 12/780,201, filed on May 14, 2010, now abandoned.
Application No. 12/780,201 is a continuation of application No. 11/920,799, now abandoned.
(60) Provisional application No. 60/840,448, filed on Aug. 28, 2006.

(51) Int. Cl. A61N 1/08

(52) U.S. Cl. 607/116

(57) ABSTRACT

An implantable connector assembly comprising a first portion having a longitudinal body which includes a transversal protrusion having therein at least one conductive socket, a generally longitudinal wire entry, at least one wire connected to the at least one conductive socket, the at least one wire entering the longitudinal body through the generally longitudinal wire entry, a second portion having a longitudinal body which includes a recess complementary to the transversal protrusion of the first portion, generally longitudinal wire entry, at least one conductive pin positioned within the recess, at least one wire connected to the at least one conductive pin, the at least one wire connected to the conducting pin entering the longitudinal body through the longitudinal wire entry and a sealing assembly. Wherein, in a connected configuration, the transversal protrusion engages the recess causing the at least one conductive pin to enter in contact with the at least one conductive socket, the sealing assembly being positioned between the transversal protrusion and the complementary recess to protect the at least one conductive pin and the at least one conductive socket from liquid infiltration.

### CROSS-REFERENCE TO RELATED APPLICATION

The present application is a continuation of U.S. Ser. No. 13/413,409 filed on Mar. 6, 2012, which is a continuation of U.S. Ser. No. 13/213,060 filed on Aug. 18, 2011, which is a continuation of U.S. Ser. No. 12/979,997 filed on Dec. 28, 2010, which is a continuation of U.S. Ser. No. 12/780,201 filed on May 14, 2010, which is a continuation of U.S. Ser. No. 11/920,799 filed on Nov. 20, 2007, which is a 371 of PCT/CA2007/001530 filed on Aug. 28, 2007, which claims the benefits of U.S. provisional patent application No. 60/840,448 filed Aug. 28, 2006, which is hereby incorporated by reference.

### TECHNICAL FIELD

The present invention relates to high density implantable connectors.

### BACKGROUND

With many surgically implanted medical devices, it is necessary to transmit electrical signals that are sensed at a remote location and carried over a flexible wire to the device as well as to deliver electrical control signals or electrical stimulation signals produced at the device to a remote location in the body via flexible wires. Furthermore, it is often necessary or desirable that a variety of configurations of sensing and stimulating components be detachable from the implanted control unit, in particular so that the control unit or individual sensors or electrodes may be replaced as needed in subsequent surgeries. Therefore, most implantable medical devices include some sort of connector that serves as the bridge between the internal electronics of the control unit and the wires that connect the control unit to the remotely located sensors, electrodes or antennae.

These connectors are often complex miniature devices and a frequent source of system failure. Reasons for connector failures may include misalignment between conductive elements, breakage of conductive elements or insulation elements, corrosion, or electrical shorts produced by fluid paths. Furthermore, because of the capillarity effect, fluids may come through the wire up to inside the connector and cause corrosion or connector shorts, leading to signal degradation. In implantable connector designs with set screws making direct electrical contact with electrodes, it is often difficult to provide good electrical isolation from surrounding body fluids and in such cases, electrostatic discharges could damage excitable tissues and/or the implanted electronics.

Therefore, there is a need for a connector for use with an implanted multi-channel device that allows reliable electrical connections between the device and a plurality of individual conducting wires while maintaining good electrical isolation between electrodes and bodily fluids. The electrical connector should be as small as possible while allowing a simple and secure connection during initial implantation and/or subsequent replacement of the control unit or of a detachable component.

### SUMMARY

The present invention relates to an implantable connector assembly comprising a first portion having a longitudinal body, including a transversal protrusion having therein at least one conductive socket, a generally longitudinal wire entry, at least one wire connected to the at least one conductive socket, the at least one wire entering the longitudinal body through the generally longitudinal wire entry, a second portion, including a longitudinal body which includes a recess complementary to the transversal protrusion of the first portion, generally longitudinal wire entry, at least one conductive pin positioned within the recess, at least one wire connected to the at least one conductive pin, the at least one wire connected to the conducting pin entering the longitudinal body through the longitudinal wire entry and a sealing assembly. Wherein, in a connected configuration, the transversal protrusion engages the recess causing the at least one conductive pin to enter in contact with the at least one conductive socket, the sealing assembly being positioned between the transversal protrusion and the complementary recess to protect the at least one conductive pin and the at least one conductive socket from liquid infiltration.

The present invention further relates to an implantable connector assembly as described above, further comprising a locking system for locking the implantable connector assembly in the connected configuration.

The present invention also relates to an implantable connector assembly as described above, wherein the locking system includes first and second locking fasteners with complimentary first and second locking inserts; the first locking fastener and the first locking insert being mounted to the first portion and the second locking fastener and the second locking insert being mounted to the second portion.

The present invention further still relates to an implantable connector assembly as described above, wherein the first and second locking inserts are respectively positioned in the proximity of the wire entry of the first and second portions and the complimentary first and second locking fasteners are respectively positioned at an end distal of the wire entry of the first and second portions.

The present invention yet further relates to an implantable connector assembly as described above, wherein the first and second portions further include a bend relief member positioned within each respective longitudinal wire entries and wherein the at least one wire connected to the at least one conductive socket enters the first portion through the bend relief member or the first portion and the at least one wire connected to the at least one conductive pin enters the second portion through the bend relief member of the second portion.

The present invention also relates to an implantable connector as described above, wherein the first and second longitudinal bodies further include a cavity contiguous to the respective longitudinal wire entries, the cavity of the first longitudinal body providing access to a connection end of the at least one conductive socket and the cavity of the second longitudinal body providing access to a connection end of the at least one conductive pin.

The present invention further relates to an implantable connector assembly as described above, wherein the first and second longitudinal bodies further include an integrated peel relief element for securing the respective bend relief member to the first and second longitudinal bodies.

### BRIEF DESCRIPTION OF THE FIGURES

Illustrative embodiments of the invention will be described by way of examples only with reference to the accompanying drawings, in which:

FIG. 1 is a perspective view of a high density implantable connector in a connected configuration according to the illustrative embodiment of the present invention;

FIG. 2 is a perspective view of the high density implantable connector of FIG. 1 in an unconnected configuration;

FIG. 3 is a cross sectional view taken along axis III-III of FIG. 1 of the high density implantable connector;

FIG. 4 is a cross sectional view taken along axis IV-IV of FIG. 2 of the high density implantable connector;

FIG. 5 is a perspective view of a second illustrative embodiment of the high density implantable connector;

FIG. 6 is a perspective view of a sealing band of the high density implantable connector of FIG. 2;

FIG. 7 is an enlarged view of portion S of FIG. 4 showing a sealing band of the high density implantable connector;

FIG. 8 is an alternative embodiment of the sealing band of FIG. 7;

FIG. 9 is an enlarged view of portion G of FIG. 4;

FIG. 10 is a perspective view of a gasket of the high density implantable connector of FIG. 3;

FIG. 11 is a perspective view of an alternative embodiment of the gasket of FIG. 10;

FIG. 12 is a perspective view of the locking mechanism of the high density implantable connector of FIG. 1;

FIG. 13 is a cross sectional view showing the insertion of a locking insert in the female portion of the high density implantable connector;

FIG. 14 is a cross sectional view showing the locking insert of FIG. 13 once inserted into the female portion of the high density implantable connector;

FIG. 15 is a cross sectional view showing the insertion of a locking screw in the female portion of the high density implantable connector;

FIG. 16 is a perspective view of the female portion of the high density implantable connector showing a first illustrative embodiment of the encapsulation;

FIG. 17 is a perspective view of the high density implantable connector showing a second illustrative embodiment of the encapsulation;

FIG. 18 is a perspective view of a sub-cutaneous tunneling device;

FIG. 19 is a cross sectional view taken along axis XIX-XIX of FIG. 18 of the sub-cutaneous tunneling device;

FIG. 20 is a perspective view of a female plug;

FIG. 21 is a perspective view of a first medical device which includes male portions of the high density implantable connector;

FIG. 22 is a perspective view of a second medical device which includes male portions of the high density implantable connector;

FIG. 23 is an exploded perspective view of the female portion of the high density implantable connector of FIG. 2;

FIG. 24 is an exploded perspective view of the male portion of the high density implantable connector of FIG. 2;

FIG. 25 is an alternate embodiment of the sealing band of FIG. 7;

FIG. 26 is a perspective view of an alternative embodiment of the female portion of the high density implantable connector having an increased bonding surface;

FIGS. 27A, 27B and 27C are perspective views of alternative embodiments of the female portion of the high density implantable connector having an integrated peel relief feature;

FIGS. 28A and 27B are perspective views of further alternative embodiments of the female portion of the high density implantable connector having a removable peel relief feature;

FIG. 29 is a perspective view of an alternative embodiment of the female portion of the high density implantable connector having a peel relief feature in the form of a lid;

FIG. 30 is a perspective view of an alternative embodiment of the locking insert;

FIG. 31 is a perspective view of an alternative embodiment of the sub-cutaneous tunneling device; and

FIG. 32 is a cross sectional view taken along axis XXXII-XXXII of FIG. 31 of the alternative embodiment of the sub-cutaneous tunneling device.

### DETAILED DESCRIPTION

Generally stated, an implantable connector, hereinafter referred to as “connector”, according to an illustrative embodiment of the present invention is used for connecting, in a removable fashion, an implanted medical device to an implantable interface which may take the form, for example, of a nerve cuff used for stimulating and/or monitoring electrical activity in nerve tissues in human beings or other creatures possessing nervous systems.

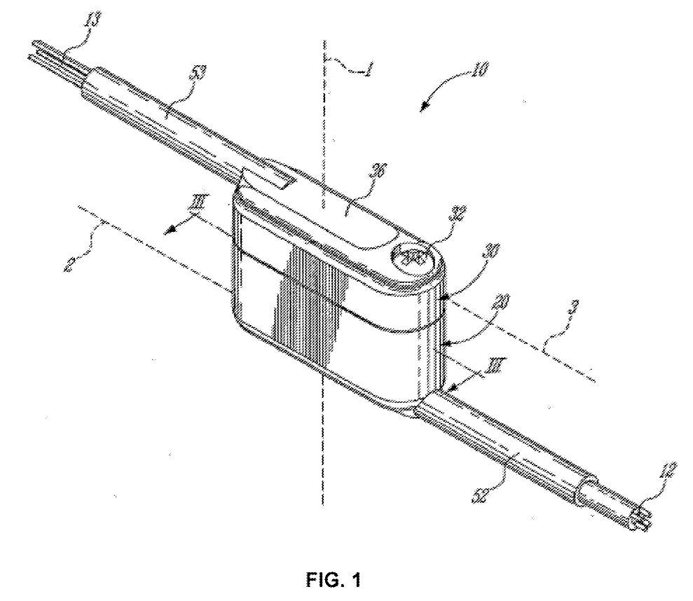

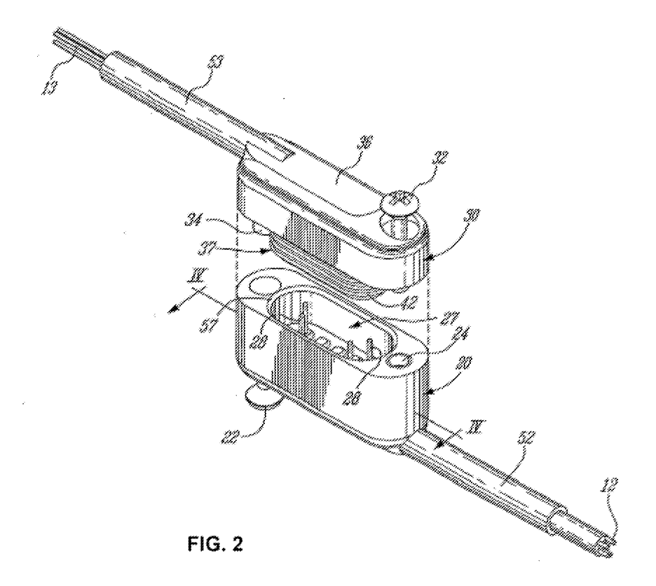

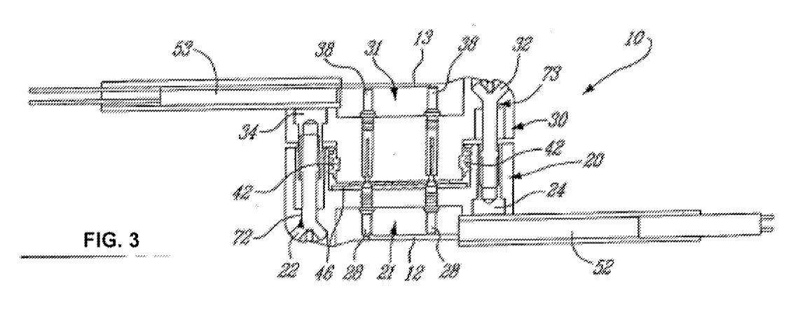

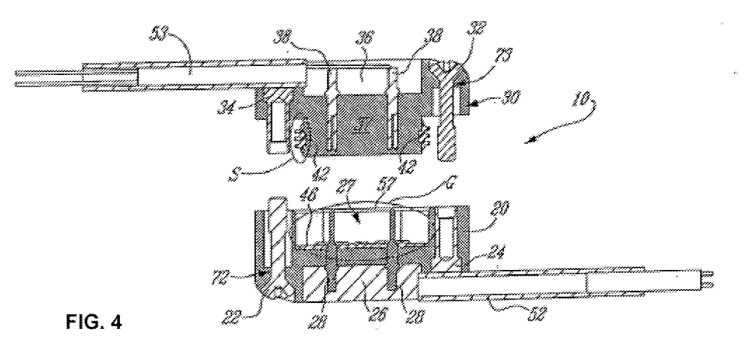

Referring to FIGS. 1-4, there is shown a non-limitative illustrative embodiment of a connector 10 having complimentary male 20 and female 30 portions in a wire-to-wire perpendicular configuration. The connector 10 is shown in FIG. 1 with its male 20 and female 30 portions joined, in FIG. 2 with its male 20 and female 30 portions separated and in respective cross-sections in FIGS. 3 and 4.

Advantageously, the surfaces of the male 20 and female 30 portions of the connector 10 may be smooth and without any pronounced irregularities as in long term implantations conjunctive tissue tends to grow in cavities or surface irregularities.

Referring to FIGS. 1 and 2, the female portion 30 includes a protrusion 37 designed to engage a complimentary recess 27 in the male portion 20 in axis 1, which is generally perpendicular to axis 2 defined by the cabling 12 of the male portion 20 and axis 3 defined by the cabling 13 of the female portion. This helps prevent the strain applied on cabling 12 and 13 from affecting the connection quality between the male 20 and female 30 portions of the connector 10. Further resistance to cabling 12, 13 bending is provided by the male portion 20 bend relief member 52 in which passes cabling 12 and by the female portion 30 bend relief member 53 in which passes cabling 13.

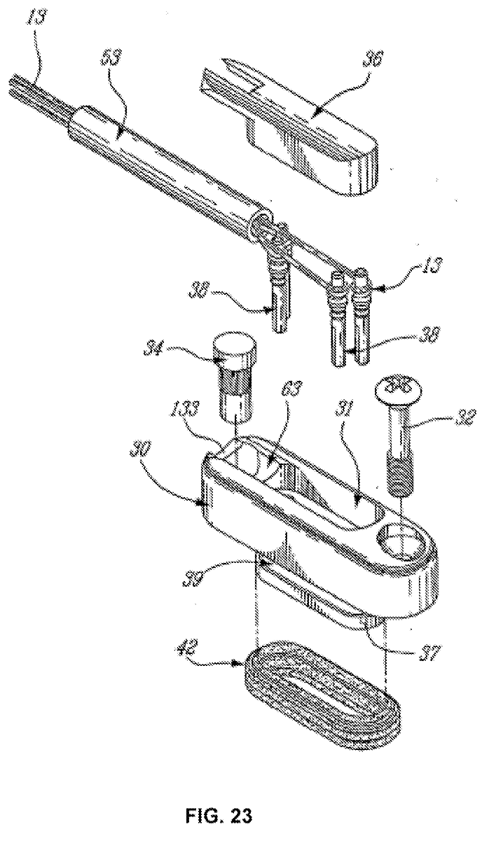

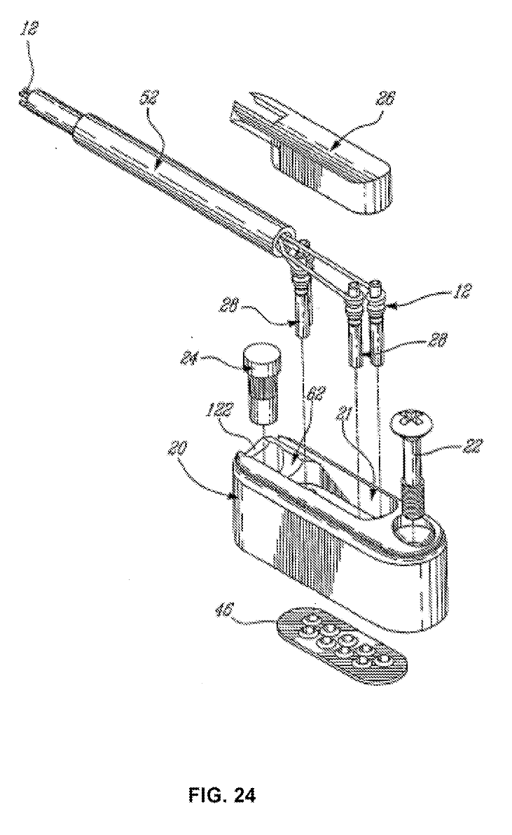

The bend relief members 52 and 53, which are positioned at respective generally longitudinal cabling entries 122 and 133 shown in FIGS. 23 and 24, help insure that the cabling 12 and 13 remain in their respective axis 2 and 3 even when under strain. It is to be understood that the cabling 12, 13 may be connected at their respective opposite ends to, for example, a nerve cuff, an implant, a control unit, a medical device, a monitoring device, etc. The bend relief members 52 and 53 will be detailed further below,

It is to be noted that the expression “generally longitudinal cable entry” is to be construed, in the present disclosure and in the appended claims as an opening configured to let cabling pass through while the cabling is generally parallel to the longitudinal portions of the implantable connector 10 according to various embodiments of the present invention.

It is also to be noted that the term “cabling” is to be construed, in the present disclosure and in the appended claims, as a wire, a plurality of wires or a cable including at least one wire.

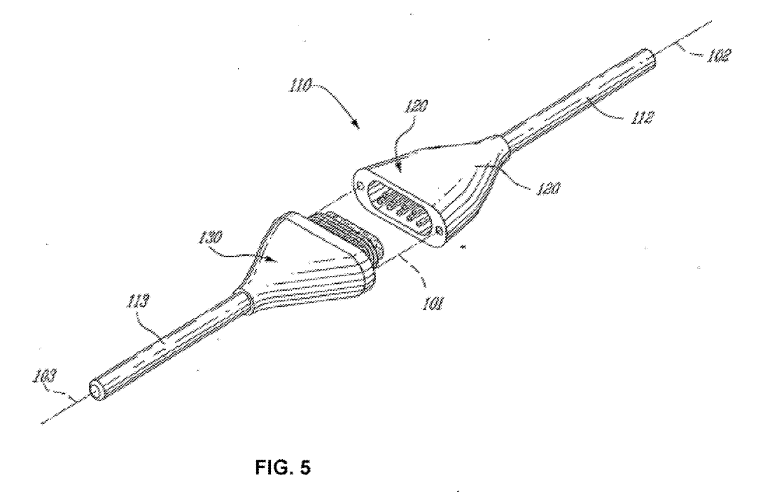

In a second illustrative embodiment, shown in FIG. 5, the connector 110 has complimentary male 120 and female 130 portions in a wire-to-wire axial configuration. In this configuration, the connection between the male 120 and female 130 portions of the connector 110 is made in axis 101 which is generally parallel to axis 102 defined by the cable 112 of the male portion 120 and to axis 103 defined by the cable 113 of the female portion 130.

Referring back to FIGS. 3 and 4 and further referring to FIGS. 23 and 24, the connector 10 includes a sealing band 42 that surrounds the protrusion 37 of the female portion 30 and a gasket 46 in the bottom of the recess 27 of the male portion 20 in order to increase its resistance to liquid infiltration. The sealing band 42 and gasket 46 will be detailed further below.

Advantageously, the protrusion 37 and complimentary recess 27 are generally oblong in shape, making the connector 10 easier to seal than if the protrusion 37 and complimentary recess 27 had a traditional D-sub profile. This is especially true for a miniature size connector 10 as the use of D-sub shaped protrusion and complimentary recess results in tight corners which could lead to a deformation of the sealing band 42 and eventually to an internal leak. The oblong shape of the protrusion 37 and complimentary recess 27 provide a more constant deformation of the sealing band 42, and thus improves the tightness of the joint between the male 20 and female 30 portions of the connector 10.

Furthermore, a chamfer 57, best seen in FIG. 2, may be created around the edge of the recess 27 of the male portion 20 in order to facilitate the insertion of the protrusion 37 of the female portion 30 and avoid potential damage to the sealing band 42 during insertion.

Providing an electrical contact between the male 20 and female 30 portions of the connector 10 are, respectively, conductive pins 28 located in the recess 27 of the male portion 20 and complimentary conductive sockets 38 located in the protrusion 37 of the female portion 30.

Advantageously, the material selected for the male 20 and female 30 parts of the connector 10 (as well as the male 120 and female 130 parts of connector 110 shown in FIG. 5) should have the following properties:
heat deflection temperature that exceeds 150° C. in order to support silicone over molding curing temperature, encapsulation epoxy curing temperature and sterilization;
water absorption that is low in order to avoid dimension variations during long exposures to body liquids; and
durometer hardness greater than 50 shore on the Durometer D scale.

A material which meets the above-mentioned requirements is PEEK-OPTIMA® polyetheretherketone, provided by INVIBIO, which is used in the development of implantable medical devices and pharmaceutical applications having blood or tissue contact for more than 30 days. It is available in a wide range of forms and may be processed via injection molding, extrusion or compression molding. This polyetheretherketone is widely used for heart valve structure, spinal cage, surgical screw, femoral implant, etc.

### Sealing Band

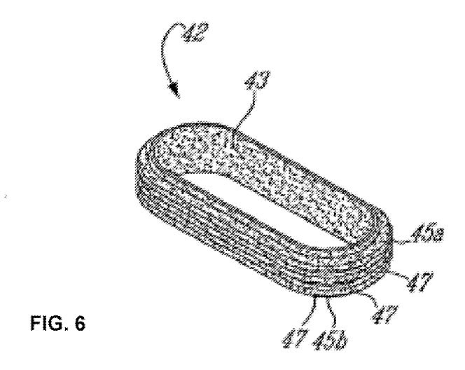

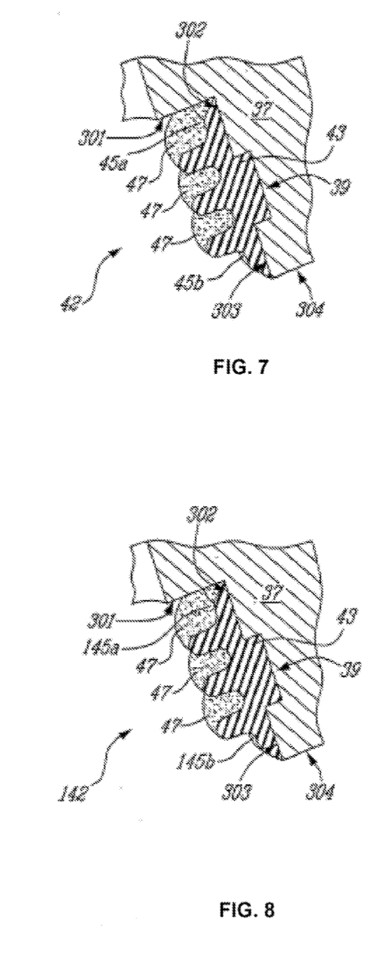

Referring to FIGS. 6 and 7, the sealing band 42, advantageously made of biocompatible silicone, for example MED-4850 silicone from NuSil, includes an anchoring member 43, laterally extending arms 45a, 45b and lips 47 to increase the barrier preventing liquid infiltration. The anchoring member 43 engages a groove 39 in the protrusion 37 of the female portion 30, as best seen in FIG. 7, providing improved grip for the sealing band 42 around the protrusion 37. Three surfaces 301, 302 and 303 of the female portion 30 of the connector 10 are available for contact with the laterally extending arms 45a, 45b; a first surface 301 perpendicular to the protrusion 37 and two surfaces 302 and 303 on the extremities of the protrusion 37.

The shape and number of anchoring member 43, laterally extending arms 45a and 45b and lips 47 depend, for example, on the space available on the protrusion 37. In the illustrative embodiment, the sealing band 42 counts one anchoring member 43, two laterally extending arms 45a, 45b and three lips 47. Thus, it is to be understood their shape and number may vary.

A first laterally extending arm 45a is in contact with surface 302, stopping short of surface 301, while laterally extending arm 45b is in contact with surface 303, stopping short of the bottom surface 304 of the protuberance 37, as best seen in FIG. 7. This configuration of the laterally extending arms 45a, 45b provides for a sealing band 42 which is independent of the height of the protrusion 37.

The sealing band 42 may be molded separately from the female portion 30 of the connector 10 and then positioned over the protrusion 37. Advantageously, the sealing band 42 may be over molded over the protrusion 37. In preparation for the over molding process, surfaces 302 and 303 of the protrusion 37, as well as the groove 39, may be roughed or surface treated with plasma for example, in order to increase the bonding between the biocompatible silicone of the sealing band 42 and the protrusion 37.

In order to help prevent the sealing band 42 from detaching from the protrusion 37, the sealing band 42 may be bonded using an adhesive. To this end, the sealing band 42 may be first over molded onto the protrusion 37, peeled off and placed back in place with an adhesive. Advantageously, a dummy protrusion (not shown) may be used to over mold the sealing band 42, the dummy protrusion having a slightly thinner and shallower groove than the groove 39 of the actual protrusion 37.

In a first alternative embodiment of the sealing band 142, shown in FIG. 8, all three surfaces 301, 302 and 303 may be used to seal the protrusion 37. In this embodiment, a first laterally extending arm 145a is in contact with both surface 302 and surface 301, while a second laterally extending arm 145b is in contact with surface 303 and stops at the edge of the bottom surface 304 of the protrusion 37.

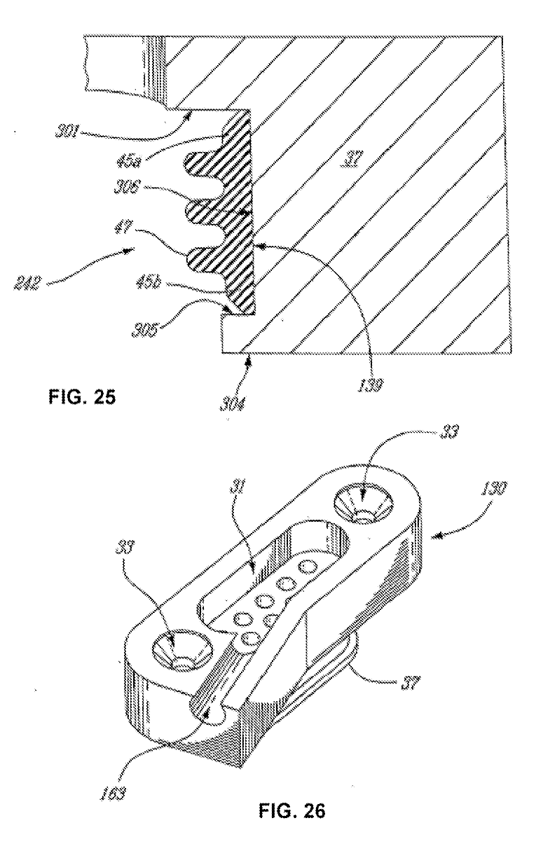

In a second alternative embodiment of the sealing band 242, shown in FIG. 25, the protrusion 37 groove 139 extends from surface 301 down to surface 305 near the bottom surface 304 and is of a width such that the sealing band 242 may be positioned completely within the groove 139, i.e. the first 45a and second 45b laterally extending arms are positioned within the groove 139. It is to be understood that the depth of the groove 139 and/or the size of the lips 47 of the sealing band 242 are chosen so that the lips 47 protrude from the groove 139. The positioning of the sealing band 242 within the groove helps improve its resistance to peeling as well as ease the installation and bonding of the sealing band 242 to the protrusion 37.

### Gasket

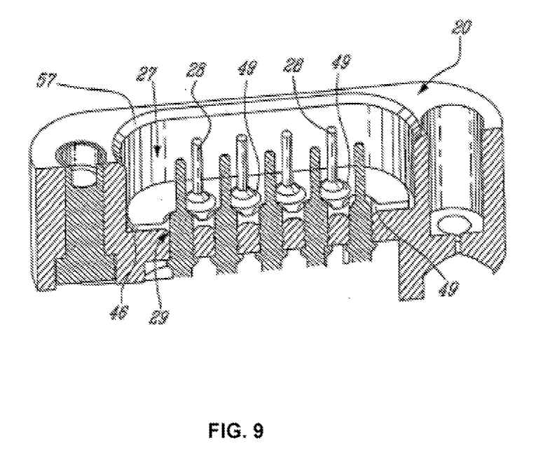

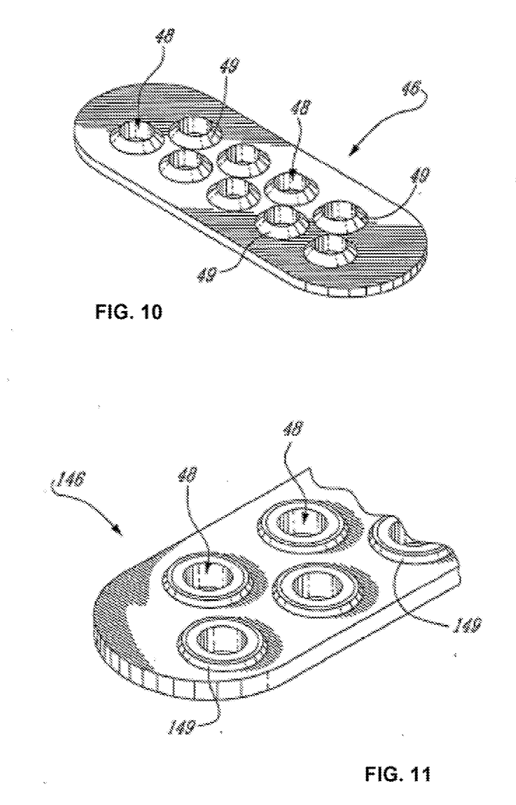

Referring to FIGS. 9 and 10, the gasket 46, may be added to the connector 10, within the recess 27 of the male portion 20 to provide a second protection layer to liquid infiltration between the male 20 and female 30 portions of the connector 10. Furthermore, the gasket 46 provides protection for the individual conductive pins 28 from electrical short-cuts, as best seen in FIG. 9.

Referring now to FIG. 10, the gasket 46, advantageously made of biocompatible silicone, for example MED-4850 silicone from NuSil, includes a number of holes 48 and associated taper projections 49, the taper projections 49 being advantageously designed larger than corresponding countersink tapers 29, shown in FIG. 9, on the male portion 20 of the connector 10. For example, the taper projections 49 may be 0.25 mm long at an angle of 45° while the countersink tapers 29 may be 0.20 mm long at an angle of 45°.

In an alternative embodiment, shown in FIG. 11, the gasket 146 includes a number of holes 48 and associated ripples 149 to increase the level of liquid tightness.

It is to be understood that the taper projections 49 and ripples 149 may be present on both sides of the gaskets 46 and 146, respectively.

### Pins and Sockets

Referring to FIGS. 3 and 4, the conductive pins 28 and sockets 38 may be advantageously press fitted in holes in the male 20 and female 30 portions, respectively, and may be made with the same material as the wires composing the cabling 12 and 13, for example stainless steel 316LV wires, to avoid possible thermocouple effects created by the junction of different materials, which may in turn lead to corrosion or signal perturbation. It is to be understood, however, that different materials may be used. Cabling 12, 13 access to the contacts of conductive pins 28 and sockets 38 is through respective cavities 21 and 31 within the male 20 and female 30 portions of the connector 10.

The wires of the cabling 12, 13 may be welded to the contacts of conductive pins 28 and sockets 38 using, for example, resistive welding or laser welding. As the resistance between two parts to be welded is important, and that resistance varies as a function of the contact area between the parts to be welded, the contacts of conductive pins 28 and sockets 38 may be flat so as to offer more contact surface.

### Type of Wire Attachment

The wires of the cabling 12, 13 may be perpendicularly welded on the contacts of the conductive pins 28 and sockets 38, respectively, with a resistance welding machine, mechanical deformation (i.e. crimping) or laser welding. Advantageously, the bodies of the contacts are bigger than the wires of the cabling 12, 13 in order to force the melting of the wires on the contacts of the conductive pins 28 and sockets 38 and not the opposite. Furthermore, the welding tip used is advantageously big enough so as to avoid heating of the tines of the conductive pins 28 and sockets 38. Too much heat may produce an annealing of the tines that may eliminate their spring effect and reduce the matting cycle capability.

For example, a micro-resistance welding machine with a closed loop control system may be used, with the current set at 260 A, a power ramp up of 4 ms, welding for 4.8 ms and a hold time for cooling down of 300 ms, while applying 5 lbs of pressure.

### Locking System

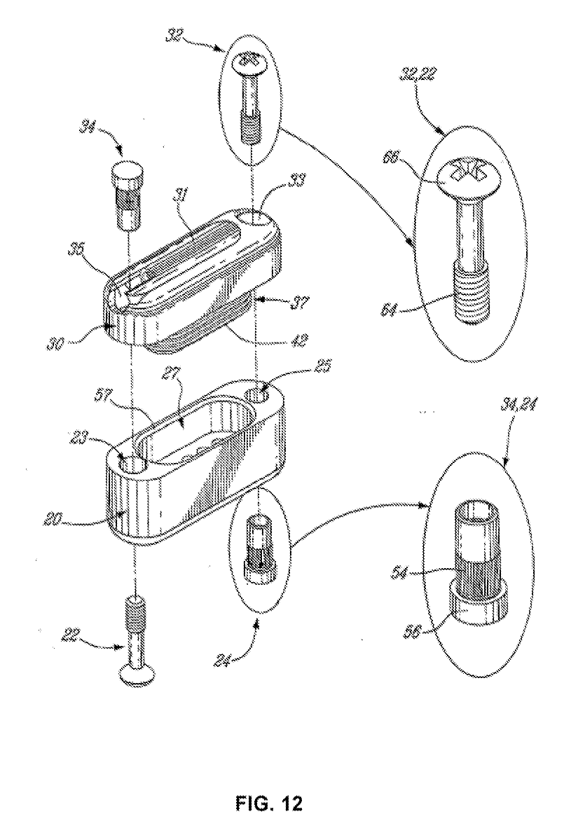

Referring to FIG. 12, the male 20 and female 30 portions of the connector 10 may be locked together using a male portion 20 locking fastener, such as a locking screw 22, with complementary female portion 30 locking insert 34 and a female portion 30 locking fastener, such as locking screw 32, with complementary male portion 20 locking insert 24. Since the recess 27 and the protrusion 37 are generally oblong, the disposition of the locking screws 22, 32 and locking inserts 24, 34 ensures that their respective male 20 and female 30 portions may be engaged in a single configuration, thus providing a mistake-proof locking system. More specifically, in the correct configuration the male portion 20 locking screw 22 engages the female portion 30 locking insert 34 while the female portion 30 locking screw 32 engages the male portion 20 locking insert 24.

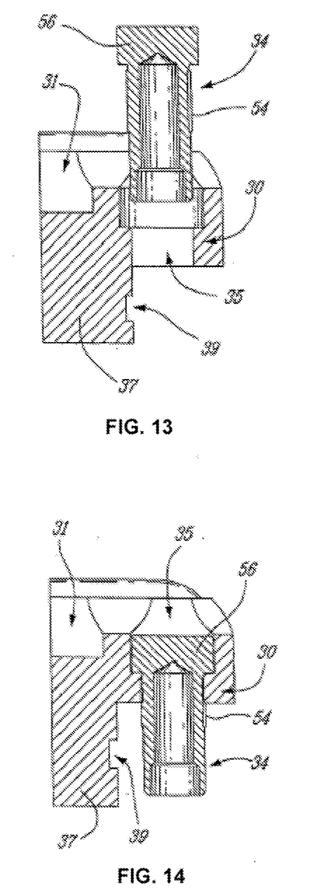

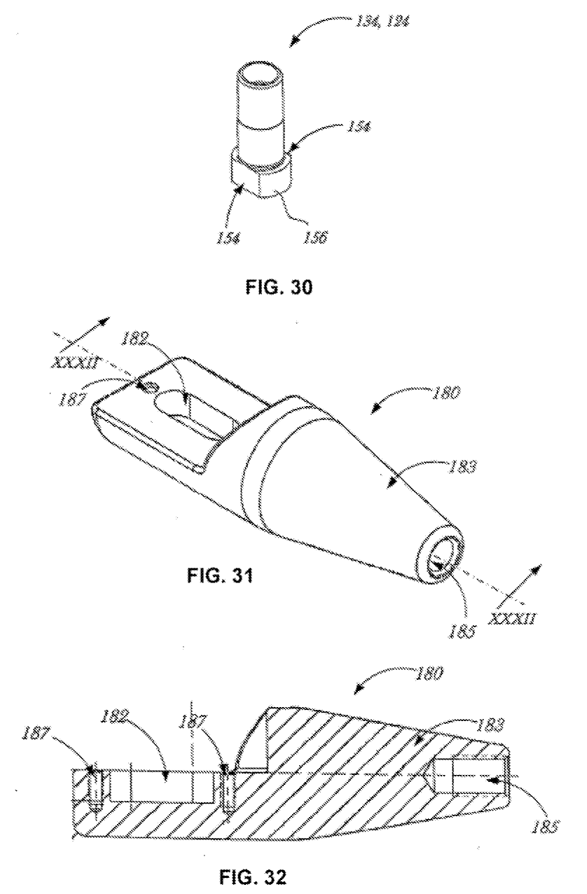

The locking inserts 24 and 34 may be press fitted in respective positioning slots 25 and 35 in the male 20 and female 30 portions. FIGS. 13 and 14 show the press fitting of the female portion 30 locking insert 34 in its positioning slot 35. It is to be understood that although the press fitting of the male portion 20 locking insert 24 in its positioning slot 25 is not shown, it is similar to that of the female portion 30 locking insert 34. Small splines 54 around the circumference of the locking inserts 24, 34, best seen in FIG. 12, help prevent their rotation within their corresponding positioning slots 25, 35 when the locking screws 32, 22 are tightened into position during the locking of the male 20 and female 30 portions. To this end, the outer diameter of the locking inserts 24, 34 at the splines 54 may be, for example, 0.1 mm larger than the diameter of the positioning slots 25, 35 in order to provide a good grip in the material of the male 20 and female 30 portions of the connector 10. In an alternative embodiment, shown in FIG. 30, the locking inserts 124, 134 may have a head 156 having one or more flat surface 154 which each interact with a corresponding flat surface within the positioning slots 25, 35 so as to prevent their rotation.

The head 56 of the locking inserts 24, 34 is advantageously loose in its corresponding positioning slot 25, 35 in order to provide a gap for bonding purposes. A bonding agent may then be applied to the head 56 of the locking inserts 24, 34 to fill the gap in their respective slots 25, 35 to further inhibit rotation.

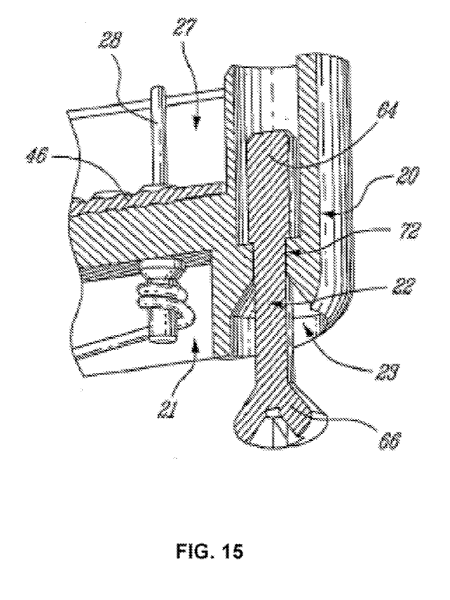

Advantageously, the locking screws 22 and 32 are trapped in their respective positioning slots 23 and 33 to avoid their loss during surgery. Threads may be machined in the narrowest section 72, 73 of the slots 23, 33, best seen in FIGS. 3 and 4, to facilitate the insertion of the locking screws 22, 32. Once the threaded section 64 of the locking screws 22 and 32 is inserted in corresponding slots 25 and 35, the locking screws 22 and 32 need to be unscrewed to be removed. The threaded section 64 may be composed of, for example, an M 1.6×0.35 thread. FIG. 15 shows the positioning of the male portion 20 locking screw 22 in its positioning slot 23. It is to be understood that although the positioning of the female portion 30 locking screw 32 in its positioning slot 33 is not shown, it is similar to that of the male portion 20 locking screw 22.

In the various figures, and in particular in FIG. 12, the locking screws 22, 32 are provided with a cross pattern Phillips #0 screw head 66. The screw head 66 may be generally oval in shape so as to avoid possible injuries caused by sharp edges. An advantage of the cross pattern screw head 66 is that it has slots which may facilitate its cleaning if obstructed with conjunctive tissue.

However, locking screws 22, 32 with a standard medical screw head with a hexagonal recess may be used as well.

Both the locking screws 22, 32 and the locking inserts 24, 34 may be made of grade 3 passivated titanium.

### Bend Relief

Referring to FIGS. 1 to 4, as well as FIGS. 23 and 24, bend relief members 52 and 53 may be added to the male 20 and female 30 portions, by inserting them in respective bonding slots 62 and 63, to avoid excessive bending of the cabling 12, 13 and to reduce the bending at the junction point of the cabling 12, 13 and their respective conductive pins 28 and sockets 38. The bend relief members 52 and 53 may be made of, for example, a silicone tube such as a #PATO7 tube provided by Alied Medical.

To bond the bend relief members 52 and 53 to their respective slots 62 and 63, a Loctite primer #7701 may first be applied on the bend relief members 52 and 53, which are then bonded to the bonding slots 62 and 63 using Loctite cyanoacrylate #4011. It is to be understood that other products or other techniques may be used in order to bond the bend relief members 52 and 53 to their respective bonding slots 62 and 63.

Once the bend relief members 52 and 53 have bonded to their respective bonding slots 62 and 63, implantable grade silicone, for example NuSil MED 4213 silicone, may be injected into the bend relief members 52 and 53 in order to create a plug, avoiding encapsulation epoxy from entering in the tube (encapsulation will be detailed below). This silicone also improves the stiffness of the bend relief members 52 and 53, increasing their bending radius. Furthermore, the silicone adhesion on the cabling 12, 13 contributes to relief bending at the junction point of the cabling 12, 13 and their respective conductive pins 28 and sockets 38.

### Bonding Surface

In an alternative illustrative embodiment, the geometry of the male 20 and female 30 portions of the connector 10 may be varied so that the bonding surface between the bend relief members 52 and 53 and their respective bonding slots 62 and 63 is increased, which in turn increases the bonding strength. Referring to FIG. 26, there is shown an example of a female portion 130 whose geometry allows for a longer bonding slot 163, which increases the bonding surface.

### Peel Relief

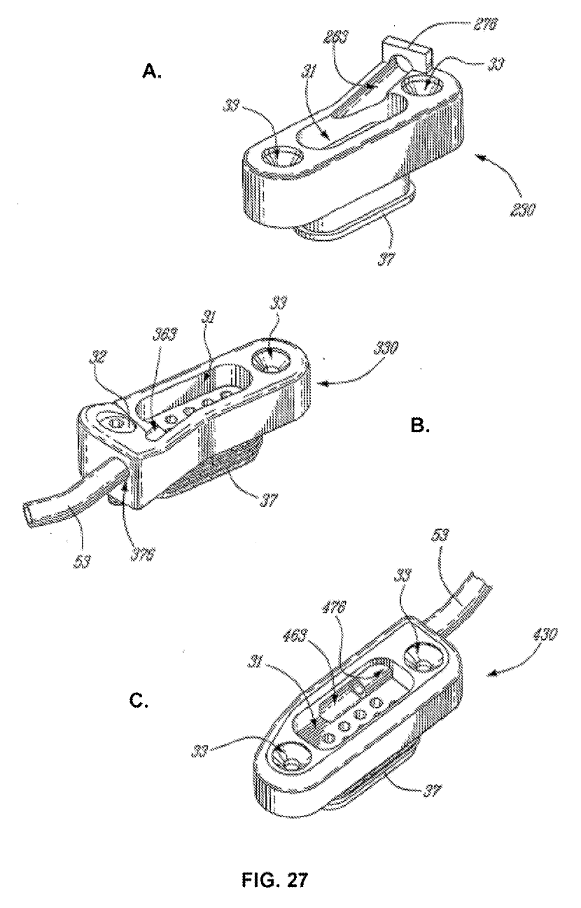

In a further illustrative embodiment, the geometry of the male 20 and female 30 portions of the connector 10 may be varied still so as to provide peel relief to the bend relief members 52 and 53. Referring to FIGS. 27A, 27B and 27C, there are shown examples of female portions 230, 330 and 430 having respective bonding slots 263, 363 and 463 and integrated peel relief member 276 or peel relief conduit 376, 476.

In use, the bend relief member 53 is introduced into the peel relief member 276 or peel relief conduit 376, 476 which help counteract the pulling force that may be exerted by the cabling 13 (not shown in FIGS. 27A, 27B and 27C) and which may result in the peeling of the bend relief member 53 from its corresponding bonding slot 263, 363 or 463. Thus, the peel relief member 276 or peel relief conduit 376, 476 ensures the integrity of the bonding between the bend relief member 53 and its corresponding bonding slot 263, 363 or 463.

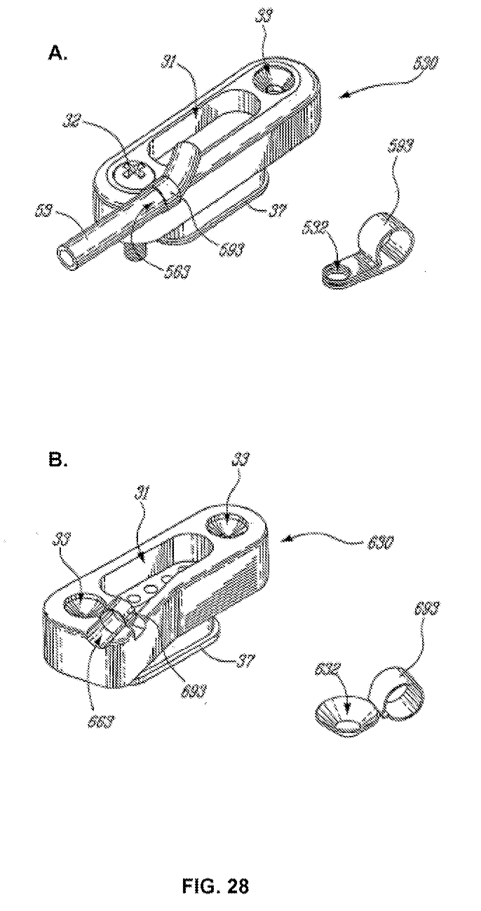

In another illustrative embodiment, a removable peel relief member may be added. Alternatively, the geometry of the male 20 and female 30 portions of the connector 10 may also be modified so as to better incorporate the removable peel relief member. Referring to FIGS. 28A and 28B, there are shown examples of female portions 530 and 630 having respective removable peel relief members 593 and 693 configured to receive therein the bend relief member 53 and hold it in respective bonding slots 563 and 663 using one of the locking screws 32 inserted through, for example, respective fixation slots 532 and 632. The removable peel relief members 593 and 693 may be made, for example, of sheet metal titanium or plastic.

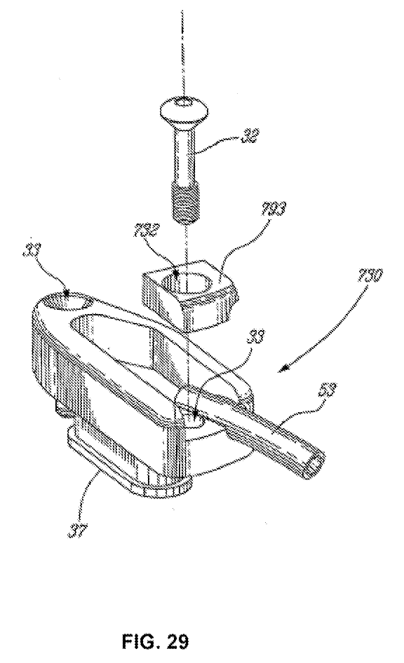

In yet another illustrative embodiment, the geometry of the male 20 and female 30 portions of the connector 10 may be varied still so as to provide for a removable lid which is complementary to the body of the male 20 or female 30 portion. Referring to FIG. 29, there is shown an example of a female portion 730 having a removable lid 793 that is complementary to the body of the female portion 730, the removable lid 793 being configured so as to fit over the positioning slot 33 closest to the bend relief member 53 and as to cover, either partially or completely, the bend relief member 53. The removable lid 793 includes a fixation slot 732 which, when the removable lid 793 is positioned onto the female portion 30 and bend relied member 53, aligns with the positioning slot 33 such that upon insertion of the locking screw 32 the bend relief member 53 is held securely in place by the removable lid 793.

### Encapsulation

Since capillary effect may bring liquid up from electrode windows in a remotely connected nerve cuff (not shown) to the junction point of the cabling 12, 13 and respective conductive pins 28 and sockets 38, it is advantageous to protect them from possible electrical short-cut due to this liquid infiltration. Moreover, encapsulation also serves as strain relief to the weld junction linking the conductive pin and wire when strain is applied on 12 or 13.

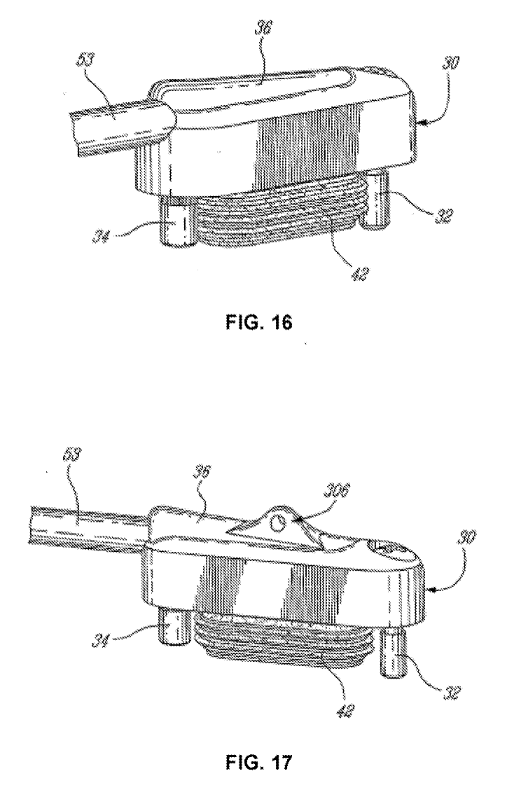

To this end, when the cabling 12 and 13 are attached to their respective conductive pins 28 and sockets 38, biocompatible casting material, for example Epoxy Epo-Tek 301 by Epoxy Technology, may be poured into their corresponding cavities 21 and 31 to prevent electrical short-cut between poles of the conductive pins 28 and sockets 38, thus forming encapsulating members 26 and 36, as best seen in FIGS. 1, 2, 4 and 16. It is to be understood that although male the male portion 20 encapsulating member 26 is not visible in all of the illustrative figures, it is similar to the female portion 30 encapsulating member 36.

In an alternative embodiment, shown in FIG. 17, an eyelet 306 may be added to the encapsulating member 36 of the female portion 30 of the connector 10 in order to allow the surgeon to attach the connector 10 inside the body of a patient. Advantageously, the dimension of the eyelet 306 corresponds to the size of suture needles used during a surgical procedure.

It is to be understood that an eyelet may also be added to encapsulating member 26 of the male portion 20 of the connector 10.

### Sub-Cutaneous Tunneling Device

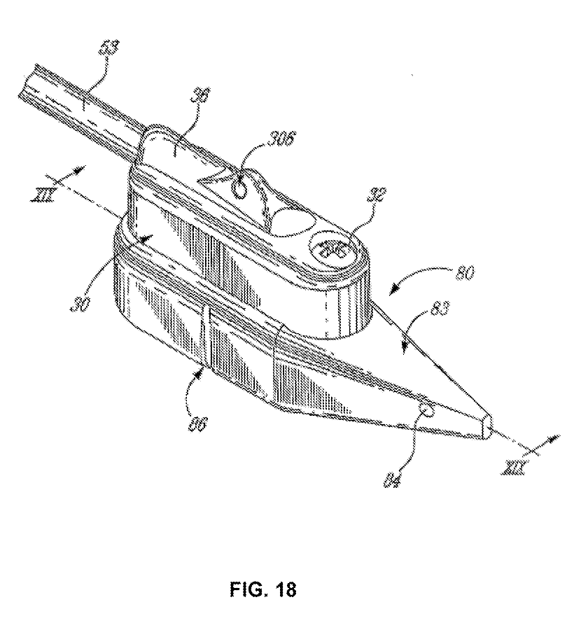

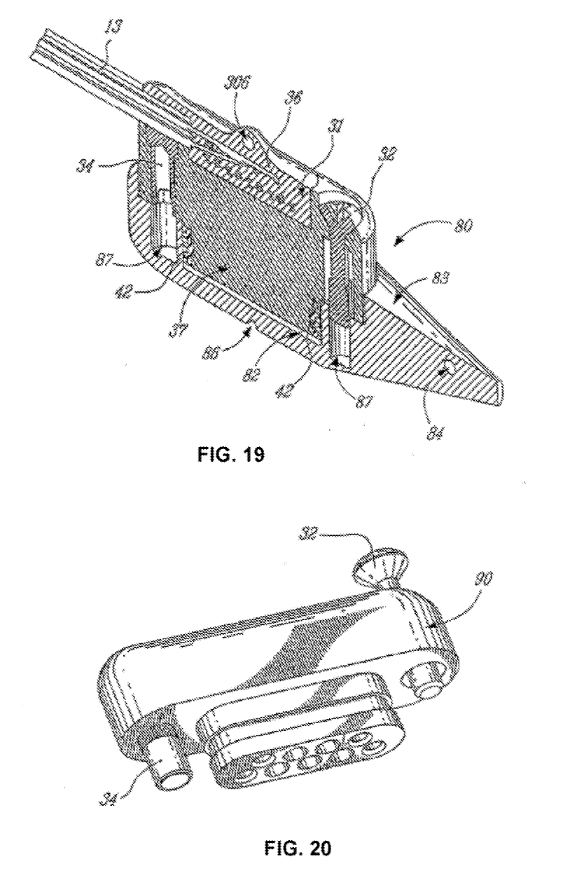

The sub-cutaneous tunneling device 80, shown in FIGS. 18 and 19, may be used to route the female portion 30 of the connector 10 under the skin of a patient from the nerve-cuff electrode implantation site to the BCU (Bio-Control Unit) implantation site. The sub-cutaneous tunneling device 80 includes a cavity 82, as best seen in FIG. 19, similar to the cavity 27 of the male portion 20 (best seen in FIG. 4) into which the female portion 30 is inserted so has to prevent liquid infiltration, one or more recess 87, optionally with a locking insert, for receiving the locking screws 22, 32, and a leading nose 83 to facilitate the displacement of the sub-cutaneous tunneling device 80 under the skin of a patient.

The sub-cutaneous tunneling device 80 may be either pushed under the skin of the patient using, for example, haemostatic pliers or, alternatively, the sub-cutaneous tunneling device 80 may be provided with an eyelet 84 to which may be tied a suture wire with which to pull the sub-cutaneous tunneling device 80.

The sub-cutaneous tunneling device 80 may further be provided with a groove 86 so as to secure the female portion 30 to the sub-cutaneous tunneling device 80 with a wire.

Furthermore, the eyelet 306, if present, may provide help in the extraction of the female portion 30 of the connector 10 from a sub-cutaneous tunneling device 80

FIGS. 31 and 32 shown an alternative embodiment of the subcutaneous tunneling device 180 which includes, similarly to the sub-cutaneous tunneling device 80, a cavity 182, one or more recess 187, optionally with a locking insert, for receiving the locking screws 22, 32, and a leading nose 183. However, the sub-cutaneous tunneling device 180 further includes an insert 185 located within the leading nose 183 configured to connect to a positioning member, for example a rod like member (not shown), which has been previously inserted under the skin of the patient from a desired end location in order to pull the sub-cutaneous tunneling device 180 to that end location. Alternatively, the insert 185 may be located at an end opposite the leading nose 183 so that the rod like member may be used to push the sub-cutaneous tunneling device 180 towards a desired end location.

The sub-cutaneous tunneling device 80, 180 may be molded, for example, with biocompatible epoxy by Epotek, PEEK-OPTIMA® polyetheretherketone, provided by INVIBIO or 316LV stainless steel.

It is to be understood that in an alternative embodiment, the subcutaneous tunneling device 80, 180 may be design so as to engage with the male portion 20 of the connector 10 instead of the female portion 30.

### Plug

Referring to FIG. 20, a female plug 90 having a similar configuration to the female portion 30 of the connector 10, but without the cabling 13, bend relief member 53, cavity 31 and conductive sockets 38, may be used to temporarily or permanently terminate an unused male portion 20 of the connector 10. Alternatively, a male plug (not shown) having a similar configuration to the male portion 20 of the connector 10, but without the cabling 12, bend relief member 52, cavity 21 and conductive pins 28, may be used to temporarily or permanently terminate an unused female portion 30 of the connector 10.

### Further Use of the Connector

Referring to FIG. 21, male 20 portions of the connector 10 may be provided to a BCU (Bio-Control Unit) 400 or other implantable device. In the illustrative example, the BCU 400 includes two male portions 20 connected to the body 410 of the BCU 400 through bend relief members 52 containing the cabling. The male portions 20 allow the connection of the BCU 400 to other devices, electrodes, nerve cuff, etc. It is to be understood that the number of male portions 20 may vary according to the desired application and that female portions 30 may be added or substituted for the male portions 20. It is also to be understood that any unused male 20 or female 30 portions may be terminated by an appropriate plug as described previously.

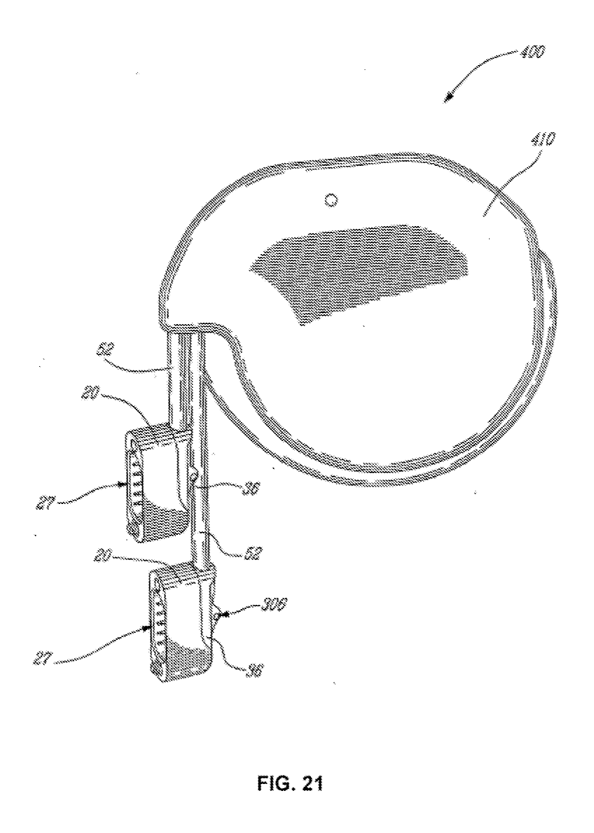

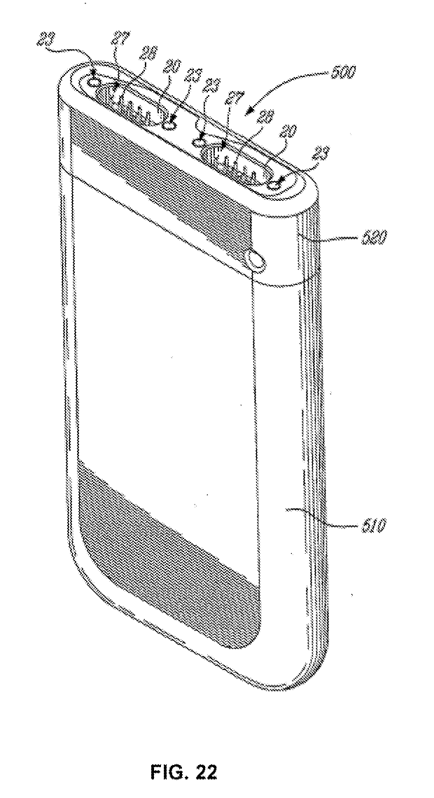

FIG. 22, shows a further example of a BCU (Bio-Control Unit) 500 having two male 20 portions of the connector 10 provided within a header 520 connected to the body 410 of the BCU 500. The male portions 20 allow the connection of the BCU 500 to other devices, electrodes, nerve cuff, etc. It is to be understood that the number of male portions 20 may vary according to the desired application and that female portions 30 may be added or substituted for the male portions 20. It is also to be understood that any unused male 20 or female 30 portions may be terminated by an appropriate plug as described previously.

Although the present invention has been described by way of particular embodiments and examples thereof, it should be noted that it will be apparent to persons skilled in the art that modifications may be applied to the present particular embodiment without departing from the scope of the present invention.

What is claimed is:

1. An implantable connector assembly, comprising:
a first portion having a longitudinal body, including:
a transversal protrusion having therein at least one conductive socket;
a generally longitudinal wire entry;
at least one wire connected to the at least one conductive socket, the at least one wire entering the longitudinal body through the generally longitudinal wire entry;
a second portion having a longitudinal body, including:
a recess complementary to the transversal protrusion of the first portion;
at least one conductive pin positioned within the recess;
at least one wire connected to the at least one conductive pin;
a sealing assembly;
wherein, in a connected configuration, the transversal protrusion engages the recess causing the at least one conductive pin to enter in contact with the at least one conductive socket, the sealing assembly being positioned between the transversal protrusion and the complementary recess to protect the at least one conductive pin and the at least one conductive socket from liquid infiltration.

2. The implantable connector assembly of claim 1, wherein the second portion further includes a generally longitudinal wire entry and wherein the at least one wire connected to the conducting pin enters the longitudinal body of the second portion through the longitudinal wire entry of the second portion.

3. The implantable connector assembly of claim 2, wherein the sealing assembly includes a sealing band surrounding the transversal protrusion.

4. The implantable connector assembly of claim 3, wherein the sealing band includes an anchoring member cooperating with a groove located on the transversal protrusion.

5. The implantable connector assembly of claim 3, wherein the sealing band is positioned within a groove located on the transversal protrusion.

6. The implantable connector assembly of claim 3, wherein the sealing band comprises at least one lip.

7. The implantable connector assembly of claim 3, wherein the sealing band comprises a pair of laterally extending arms.

8. The implantable connector assembly of claim 2, wherein the sealing assembly includes a gasket positioned at a bottom end of the recess.

9. The implantable connector assembly of claim 8, wherein the gasket comprises at least one hole for letting through the at least one conductive pin, the at least one hole having an associated taper projection.

10. The implantable connector assembly of claim 9, wherein the bottom end of the recess includes at least one countersink taper corresponding with the at least one taper projection.

11. The implantable connector assembly of claim 8, wherein the gasket comprises at least one hole for letting through the at least one conductive pin, each hole having an associated plurality of ripples.

12. The implantable connector assembly of claim 2, further comprising a locking system for locking the implantable connector assembly in the connected configuration.

13. The implantable connector assembly of claim 12, wherein the locking system includes first and second locking fasteners with complimentary first and second locking inserts; the first locking fastener and the first locking insert being mounted to the first portion and the second locking fastener and the second locking insert being mounted to the second portion.

14. The implantable connector assembly of claim 13, wherein the first and second locking inserts are respectively positioned in the proximity of the wire entry of the first and second portions and the complimentary first and second locking fasteners are respectively positioned at an end distal of the wire entry of the first and second portions.

15. The implantable connector assembly of claim 13, wherein the first and second locking fasteners include locking screws.

16. The implantable connector assembly of claim 15, wherein the locking screws provided a head having a generally oval shape.

17. The implantable connector assembly of claim 15, wherein the locking screws include a cross pattern Phillips #0 head.

18. The implantable connector assembly of claim 15, wherein the locking screws include a standard medical screw head.

19. The implantable connector assembly of claim 13, wherein the locking inserts include longitudinal splines.

20. The implantable connector assembly of claim 13, wherein the locking inserts include at least one flat surface.

21. The implantable connector assembly of claim 2, wherein the protrusion and the recess are generally oblong in shape.

22. The implantable connector assembly of claim 2, wherein the second portion includes a chamfer around the edge of an open end of the recess.

23. The implantable connector assembly of claim 2, wherein the first and second portions further include a bend relief member positioned within each respective longitudinal wire entries and wherein the at least one wire connected to the at least one conductive socket enters the first portion through the bend relief member or the first portion and the at least one wire connected to the at least one conductive pin enters the second portion through the bend relief member of the second portion.

24. The implantable connector assembly of claim 23, wherein the bend relief member is in the form of a tube.

25. The implantable connector assembly of claim 24, wherein the bend relief member is filled with implantable grade silicone.

26. The implantable connector assembly of claim 25, wherein the implantable grade silicone includes NuSil MED 4213 silicone.

27. The implantable connector assembly of claim 23, wherein the first and second longitudinal bodies further include an integrated peel relief element for securing the respective bend relief member to the first and second longitudinal bodies.

28. The implantable connector assembly of claim 23, wherein the first and second longitudinal bodies further include a removable peel relief element for securing the respective bend relief member to the first and second longitudinal bodies.

29. The implantable connector assembly of claim 2, wherein the first and second longitudinal bodies further include a cavity contiguous to the respective longitudinal wire entries, the cavity of the first longitudinal body providing access to a connection end of the at least one conductive socket and the cavity of the second longitudinal body providing access to a connection end of the at least one conductive pin.

30. The implantable connector assembly of claim 29, wherein the cavity of the first and second longitudinal bodies is filed with a biocompatible material forming an encapsulating member.

31. The implantable connector assembly of claim 30, wherein the biocompatible material includes Epoxy Epo-Tek 301.

32. The implantable connector assembly of claim 30, wherein the encapsulating member includes an eyelet.

33. The implantable connector assembly of claim 32, wherein the dimension of the eyelet generally corresponds to the size of suture needles used during a surgical procedure.

34. The implantable connector assembly of claim 2, further comprising a tunneling device having a longitudinal body.

35. The implantable connector assembly of claim 34, wherein the longitudinal body of the tunneling device includes a recess complementary to the transversal protrusion of the first portion.

36. The implantable connector assembly of claim 35, wherein the tunneling device includes an eyelet.

37. The implantable connector assembly of claim 35, wherein the longitudinal body of the tunneling device includes a generally transversal groove.

38. The implantable connector assembly of claim 35, wherein the tunneling device includes a leading nose.

39. The implantable connector assembly of claim 38, wherein the leading nose includes an insert configured to connect to a positioning member.

40. The implantable connector assembly of claim 38, wherein the longitudinal body of the tunneling device includes at an end opposite the leading nose an insert configured to connect to a positioning member.

41. The implantable connector assembly of claim 34, wherein the tunneling device includes a transversal protrusion having therein at least one conductive socket.

42. The implantable connector assembly of claim 41, wherein the tunneling device includes an eyelet.

43. The implantable connector assembly of claim 41, wherein the longitudinal body of the tunneling device includes a generally transversal groove.

44. The implantable connector assembly of claim 41, wherein the tunneling device includes a leading nose.

45. The implantable connector assembly of claim 44, wherein the leading nose includes an insert configured to connect to a positioning member.

46. The implantable connector assembly of claim 44, wherein the longitudinal body of the tunneling device includes at an end opposite the leading nose an insert configured to connect to a positioning member.
